(19) United States
(12) Patent Application Publication
(10) Pub. No.: US 2025/0134845 A1
(43) Pub. Date: May 1, 2025

(54) Fumaric Acid Esters, Inhalation Administration Methods, Pharmaceutical Uses and Compositions

(71) Applicants: Emory University, Atlanta, GA (US); Arizona Board of Regents on behalf of the University of Arizona, Tuscon, AZ (US)

(72) Inventors: Louise Hecker, Atlanta, GA (US); Kosuke Kato, Atlanta, GA (US); Heidi M. Mansour, Tuscon, AZ (US); Priyadarshini Muralidharan, Tucson, AZ (US)

(21) Appl. No.: 18/838,780

(22) PCT Filed: Feb. 15, 2023
(86) PCT No.: PCT/US2023/013113

§ 371 Date: Aug. 15, 2024

Related U.S. Application Data
(60) Provisional application No. 63/310,195, filed on Feb. 15, 2022.

(51) Int. Cl. A61K 31/225; A61K 9/00; A61K 31/4418; A61K 31/496; A61K 45/06; A61P 11/00

(52) U.S. Cl. CPC A61K 31/225; A61K 9/0078; A61K 9/008; A61K 31/4418; A61K 31/496; A61K 45/06; A61P 11/00

(57) ABSTRACT

This disclosure relates to uses of fumaric acid, fumaric acid esters, dimethyl fumarate, monomethyl fumarate ester, salt, or derivatives thereof by pulmonary administration in the treatment, prevention, or reversal of fibrosis e.g., pulmonary fibrosis, idiopathic pulmonary fibrosis (IPF). In certain embodiments, this disclosure relates to treating a subject with fumaric acid, fumaric acid esters, dimethyl fumarate, monomethyl fumarate ester, salt, or derivatives inhibiting TGF-mediated pro-fibrotic phenotypes. In certain embodiments, the subject is a human of an advanced age.

### CROSS-REFERENCE TO RELATED APPLICATIONS

This application claims the benefit of U.S. Provisional Application No. 63/310,195 filed Feb. 15, 2022. The entirety of this application is hereby incorporated by reference for all purposes.

### STATEMENT REGARDING FEDERALLY SPONSORED RESEARCH OR DEVELOPMENT

This invention was made with government support under AG054766 awarded by the National Institutes of Health. The government has certain rights in this invention.

### BACKGROUND

Human fibrotic disorders affect many organ systems, and significant number of deaths are attributable to disorders that are characterized by varying degrees of fibrosis. The most severe form of lung fibrosis is idiopathic pulmonary fibrosis (IPF), a fatal and progressive disorder common in the elderly. IPF is characterized by excessive scar tissue formation and irreversible destruction of the lung parenchyma resulting in gas-exchange abnormalities and ultimately respiratory failure. There is a need for therapies that can prevent or promote the resolution of established lung fibrosis.

Oxidative stress is characterized as an imbalance between reactive oxygen species (ROS) production and antioxidant capacity. Defective antioxidant responses are implicated in the pathogenesis of IPF.

Hecker et al. report the reversal of persistent fibrosis in aging by targeting Nox4-Nrf2 redox imbalance. Sci. Transl. Med. 2014, 6, 231ra247.

Grzegorzewska et al. report dimethyl fumarate ameliorates pulmonary arterial hypertension and lung fibrosis by targeting multiple pathways. Sci. Rep. 2017, 7, 41605.

Kato et al. report the efficacy of nintedanib in aging models of pulmonary fibrosis. Eur. Respir. J. 2021, 58, 2100759.

Pinto et al. report inhalation of dimethyl fumarate-encapsulated solid lipid nanoparticles attenuate clinical signs of experimental autoimmune encephalomyelitis and pulmonary inflammatory dysfunction in mice. Clin Sci (Lond), 2022, 136 (1): 81-101.

See also U.S. Pat. Nos. 11,484,530, 10,576,055, and 9,422,226.

References cited herein are not an admission of prior art.

### SUMMARY

This disclosure relates to uses of fumaric acid, fumaric acid esters, dimethyl fumarate, monomethyl fumarate ester, salt, or derivatives thereof by pulmonary administration in the treatment, prevention, or reversal of fibrosis e.g., pulmonary fibrosis, idiopathic pulmonary fibrosis (IPF). In certain embodiments, this disclosure relates to treating a subject with fumaric acid, fumaric acid esters, dimethyl fumarate, monomethyl fumarate ester, salt, or derivatives inhibiting TGF-mediated pro-fibrotic phenotypes and established pro-fibrotic phenotypes. In certain embodiments, the subject is a human of an advanced age.

In certain embodiments, this disclosure relates to methods of treating or preventing lung fibrosis comprising administering to the lungs an effective amount of a fumaric acid ester or salt thereof to a subject in need thereof. In certain embodiments, the fumaric acid ester is a dialkyl ester such as dimethyl fumarate. In certain embodiments, the fumaric acid ester is a monoalkyl ester such as monomethyl fumarate or salt thereof.

In certain embodiments, administration is by inhalation of an aerosol of a fumaric acid ester, dimethyl fumarate, monomethyl fumarate ester or salt thereof in the pulmonary airway. In certain embodiments, administration is by inhalation of a fumaric acid ester, dimethyl fumarate, monomethyl fumarate ester or salt thereof through the mouth and/or nose.

In certain embodiments, administration is by a metered-dose inhaler. In certain embodiments, administration is by a single or multiple dose dry powder inhaler. In certain embodiments, administration is by a nebulizer.

In certain embodiments, this disclosure relates to pharmaceutical compositions, containers, and kits comprising a fumaric acid ester, dimethyl fumarate, monomethyl fumarate ester or salt thereof for use in pulmonary administration and optionally comprising another active agent. In certain embodiments, the container is a pressurized or unpressurized container. In certain embodiments, the container is a manual pump spray, inhaler, meter-dosed inhaler, dry powder inhaler, nebulizer, vibrating mesh nebulizer, jet nebulizer, or ultrasonic wave nebulizer.

### BRIEF DESCRIPTION OF THE SEVERAL VIEWS OF THE DRAWINGS

FIGS. 1A-1D show data indicating dimethyl fumarate (DMF) treatment inhibits pro-fibrotic phenotypes in IPF lung fibroblasts.

FIG. 1A shows data where primary fibroblasts were isolated from the lung of a patient with biopsy-proven IPF. IPF lung fibroblasts were treated with DMF (1 μM) or vehicle (DMSO) followed by treatment TGF-b (2 ng/ml). H₂O₂ levels were evaluated at 24 h by Amplex™ Red assay.

FIG. 1B shows data where whole-cell lysates were assessed for collagen-1 expression at 48 h by Western blot; densitometric analyses are shown.

FIG. 1C shows data where IPF lung fibroblasts were treated with DMF (1 μM) or vehicle (DMSO). Culture supernatant was evaluated for H₂O₂ levels at 24 h by Amplex™ Red assay.

FIG. 1D shows data where whole-cell lysates were assessed for collagen-1 expression at 24 h by Western blot: densitometric analyses are shown.

FIGS. 2A-2D show data indicating aged-injured mice exhibit (nuclear factor erythroid 2-related factor 2 (Nrf2) deficiency associated with oxidative stress and lung fibrosis.

FIG. 2A shows data on lung protein Nrf2 expression assessed by densitometric analysis.

FIG. 2B shows data on the level of lung oxidized glutathione analyzed by a quantitative oxidized glutathione assay.

FIG. 2C shows data on lung protein fibronectin assessed by densitometric analysis.

FIG. 2D shows data on collagen-1 (Col-1) assessed by densitometric analysis.

FIG. 3A-3D shows data indicating intranasal (but not oral gavage) administration of DMF promotes reversal of age-associated established lung fibrosis.

FIG. 3A shows a schematic diagram illustrating age-dependent persistent lung fibrosis and treatment protocol. C57BL/6 aged (18 month) female mice received an intratracheal instillation of bleomycin (0.02875 U/mouse). DMF was administered daily by oral gavage (240 μg/150 μL in sterile PBS/mouse) or intranasal instillation (80 μg/50 μL in sterile PBS/mouse) from 3-6 weeks post-injury. Lung tissue was assessed by hematoxylin and eosin (H&E) staining for histopathology and Masson's trichrome blue staining for collagen.

FIG. 3B show data on lung protein extract assessed for fibronectin. Oral-vehicle (n=6); oral-DMF (n=5); intranasal-vehicle (n=4); intranasal-DMF (n=6).

FIG. 3C shows data on collagen-1 expression by densitometric analysis.

FIG. 3D shows data indicating resolution of fibrosis analyzed by quantitative hydroxyproline assay. Oral-vehicle (n=5); oral-DMF (n=5); intranasal-vehicle (n=5); intranasal-DMF (n=6).

FIG. 4A-4D shows data indicating intranasal (but not oral gavage) administration of DMF restores lung Nrf2 levels in aged-injured mice.

FIG. 4A shows data on blood reactive oxygen species (ROS) levels analyzed by Amplex™ Red assay. Oral-DMF (n=7); intranasal-DMF (n=6).

FIG. 4B shows data on lung protein extract assessed for Nrf2 expression by densitometric analysis. Oral-vehicle (n=7); oral-DMF (n=7); intranasal-vehicle (n=4); intranasal-DMF (n=4).

FIG. 4C shows data on the level of lung oxidized glutathione analyzed by a quantitative oxidized glutathione assay. Oral-vehicle (n=5); oral-DMF (n=5); intranasal-vehicle (n=3); intranasal-DMF (n=4).

FIG. 4D shows data on the level of lung lipid peroxidation analyzed by quantitative lipid peroxidation assay. Oral-vehicle (n=5); oral-DMF, (n=5); intranasal-vehicle (n=4); intranasal-DMF (n=3).

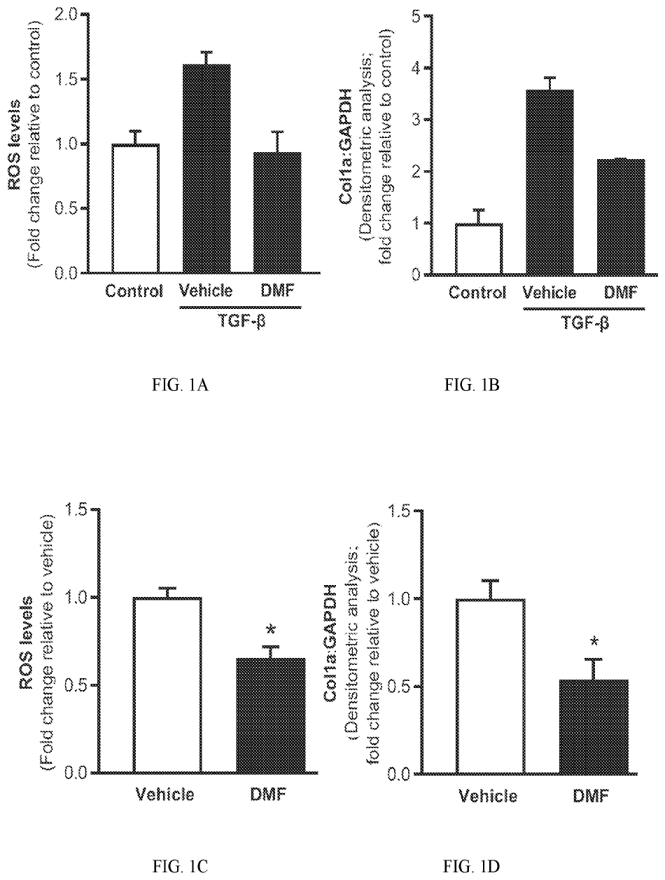

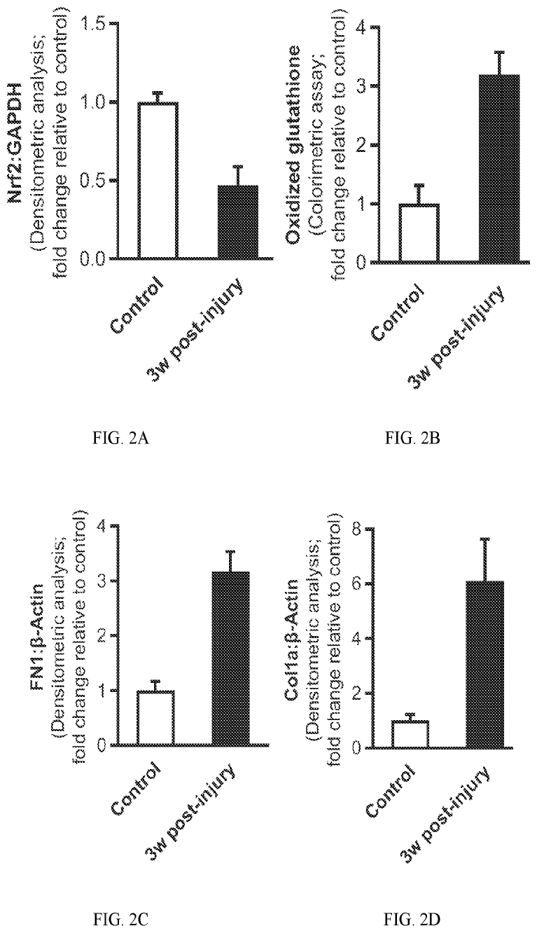

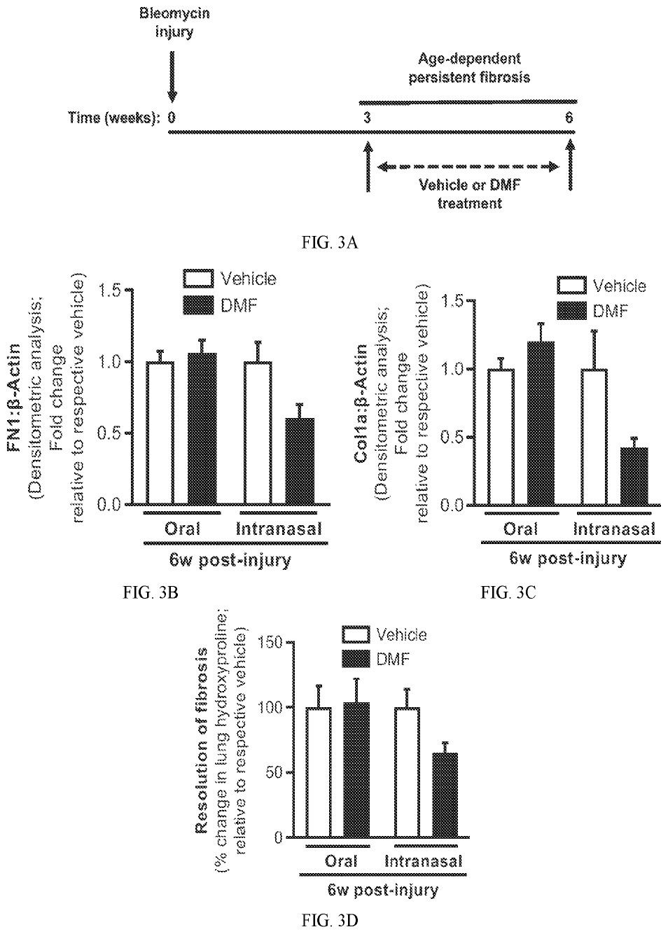

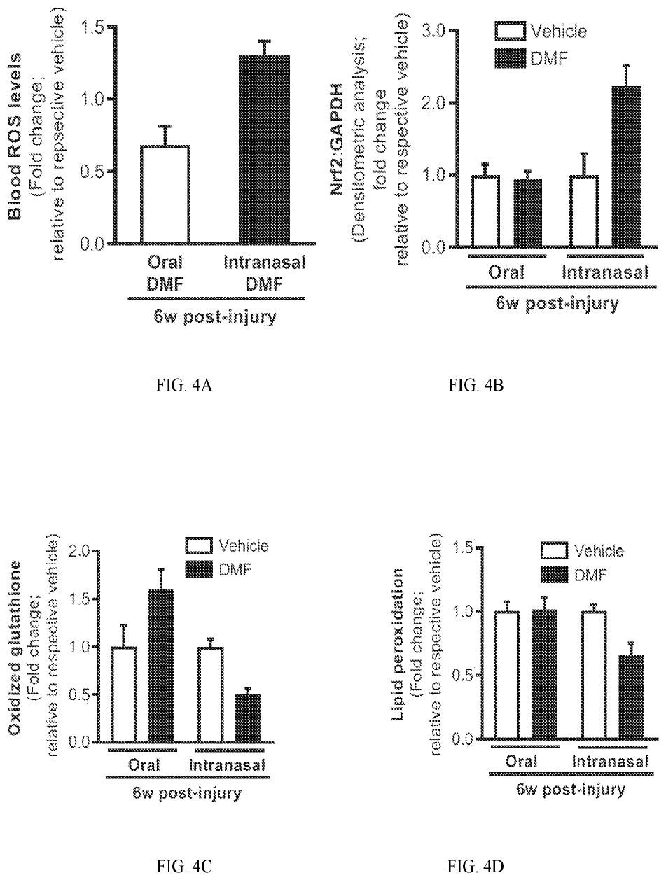

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

An “embodiment” of this disclosure refers to an example and infers that the example is not necessarily limited to the example. Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, have the meaning ascribed to them in U.S. Patent law in that when applied to methods and compositions encompassed by the present disclosure refers to the idea of excluding certain prior art element(s) as an inventive feature of a claim, but which may contain additional composition components or method steps, etc., that do not materially affect the basic and novel characteristic(s) of the compositions or methods, compared to those of the corresponding compositions or methods disclosed herein.

As used herein, the term “about” is synonymous with the term “approximately.” Illustratively, the use of the term “about” indicates that a value includes values slightly outside the cited values. Variation may be due to conditions such as experimental error, manufacturing tolerances, variations in equilibrium conditions, and the like. In some embodiments, the term “about” includes the cited value plus or minus 5% or 10%. In all cases, where the term “about” has been used to describe a value, it should be appreciated that this disclosure also supports the exact value.

“Subject” refers to any animal, preferably a human patient, livestock, rodent, monkey, or domestic pet.

As used herein, the terms “treat” and “treating” are not limited to the case where the subject (e.g., patient) is cured and the disease is eradicated. Rather, embodiments of the present disclosure also contemplate treatment that merely reduces symptoms, and/or delays disease progression.

As used herein, the term “in combination with,” when referring to two or more compounds, agents, or additional active pharmaceutical ingredients, means the administration of two or more compounds, agents, or active pharmaceutical ingredients to a subject or human patient prior to, concurrent with, or subsequent to each other such that they are contained/circulating in the patient at the same time, e.g., considering half-lives of the agents.

“Pulmonary fibrosis” refers to thickening or scarring of lung tissue. The normally thin, lacy walls of the air sacs in the lungs are no longer thin and lacy, but get thick, stiff and/or scarred, i.e., fibrotic. “Idiopathic pulmonary fibrosis” refers to someone with pulmonary fibrosis for unknown reasons, e.g., a subject that has pulmonary fibrosis without a diagnosis of cystic fibrosis or caused by exposure to lung toxin, coal miner, or cigarette smoker.

“Bronchiectasis” refers to a condition where the walls of the bronchi are thickened which can result in periodic flare-ups of breathing difficulties, also referred to as exacerbations. Cylindrical (tubular) bronchiectasis is characterized by cylinder-shaped bronchi/bronchioles. Cylindrical bronchiectasis is a morphologic type of bronchiectasis where there is smooth uniform enlargement of bronchi with loss of the normal distal tapering of the airways without focal outpouchings. Bronchial dilatation is typically evaluated in relation to the accompanying pulmonary artery. A broncho to arterial ratio greater than 1:1 is typically considered abnormal. Normal bronchi are narrower in diameter the further they are from the lung hilum. Lack of normal bronchial tapering over 2 cm in length, distal from an airway bifurcation, is a sign of bronchiectasis. Varicose bronchiectasis bronchi are irregular, and the airways may be wide or constricted. In cystic bronchiectasis, cysts can occur in the subpleural areas, when they typically represent paraseptal emphysema, bullae, or honeycombing. Bronchiectasis is typically a chronic respiratory condition, characterized by frequent cough and shortness of breath due to a range of conditions that include inherited mucociliary defects, inhalational airway injury, immunodeficiency states and prior respiratory infections. Bronchiectasis is characterized as a thickening and dilation of the walls of the bronchi from inflammation, infection, or other etiologies which result in the inability to clear mucus from the airway. Affected individuals are then more susceptible to repeated lung infections. Bronchiectasis is commonly found in individuals with cystic fibrosis. Cystic fibrosis is typically diagnosed in human patients having mutations in the cystic fibrosis transmembrane conductance regulator (CFTR) gene. CF patients are typically diagnosed with and persistent pulmonary infections, elevated sweat chloride, and pancreatic insufficiency. In certain embodiments, elevated sweat chloride is in a concentration above 30 or 60 millimoles per liter (mEq/L).

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing a oxygen atom with a sulfur atom, replacing an amino group with a hydroxyl group, replacing a nitrogen with a protonated carbon (CH) in an aromatic ring, replacing a bridging amino group (—NH—) with an oxy group (—O—), or vice versa. The derivative may be a prodrug or a metabolite. Derivatives may be prepared by any variety of synthetic methods or appropriate adaptations presented in synthetic or organic chemistry textbooks, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

The term “prodrug” refers to an agent that is converted into a biologically active form in vivo, i.e., a “metabolite.” The conversion of an ester to a carboxylic acid in vivo is a common metabolite. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug metabolite by various mechanisms, including enzymatic processes and metabolic hydrolysis. Typical prodrugs are pharmaceutically acceptable esters. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO2Rb, —C(═O)Ra, —C(═O)ORa, —C(═O)NRaRb, —OC(═O)NRaRb, —ORa, —SRa, —SORa, —S(═O)2Ra, —OS(═O)2Ra and —S(═O)2ORa. Ra and Rb in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, and heteroarylalkyl

### Methods of Use

This disclosure relates to uses of fumaric acid, a fumaric acid ester, dimethyl fumarate, monomethyl ester, salt, or derivatives thereof in the treatment, prevention, or reversal of fibrosis e.g., pulmonary fibrosis, idiopathic pulmonary fibrosis (IPF). In certain embodiments, this disclosure relates to treating a subject by inhalation to the lung with dimethyl fumarate inhibiting pro-fibrotic phenotypes and reversing IPF lung fibroblasts.

In certain embodiments, this disclosure relates to methods of treating or preventing lung fibrosis comprising administering by inhalation to the lung an effective amount of fumaric acid, a fumaric acid ester, or salt thereof to a subject in need thereof. In certain embodiments, the fumaric acid ester is a dialkyl ester such as dimethyl fumarate. In certain embodiments, the fumaric acid ester is a monoalkyl ester such as monomethyl fumarate or salt thereof.

In certain embodiments, administration is by inhalation of an aerosol of fumaric acid, a fumaric acid ester, dimethyl fumarate, monomethyl fumarate ester or salt thereof in the pulmonary airway. In certain embodiments, administration is by inhalation of a fumaric acid ester, dimethyl fumarate, monomethyl fumarate ester or salt thereof through the mouth and/or nose.

In certain embodiments, administration is by a metered-dose inhaler. In certain embodiments, administration is by a single or multiple dose dry powder inhaler. In certain embodiments, administration is by a nebulizer. In certain embodiments, is a jet nebulizer driven by compressed air. In certain embodiments, the nebulizer is an ultrasonic nebulizer having a piezoelectric transducer for creating droplets from a liquid reservoir. In certain embodiments, the nebulizer is vibrating mesh nebulizer having perforated membranes actuated by an annular piezo element that vibrates in resonant bending mode.

In certain embodiments, administration is by intratracheal instillation, e.g., using a syringe.

In certain embodiments, administration is by inhalation of fumaric acid, a fumaric acid ester, dimethyl fumarate, monomethyl fumarate ester or salt thereof through a nostril or the mouth.

In certain embodiments, the subject is a human subject greater than 55, 60, 65, or 70 years of age.

In certain embodiments, fumaric acid, the fumaric acid ester, dimethyl fumarate, monomethyl fumarate ester or salt thereof is administered daily or twice daily for more than one, two, three, four, five, or six weeks, or more than two months.

In certain embodiments, the subject is diagnosed with pulmonary fibrosis, bronchiectasis, or cystic fibrosis.

In certain embodiments, the subject is diagnosed with bacterial infection or viral infection.

In certain embodiments, fumaric acid, the fumaric acid ester, dimethyl fumarate, monomethyl fumarate ester or salt thereof is administered in combination with another active agent such as a bronchodilator, corticosteroid, antimuscarinic, antibiotic, nintedanib, pirfenidone, or combinations thereof.

In certain embodiments, the bronchodilator is a beta-2 agonist, such as salbutamol, salmeterol, formoterol and vilanterol or an anticholinergic, such as ipratropium, tiotropium, aclidinium, or glycopyrronium, or an antimuscarinic such as atropine or scopolamine, or theophylline.

In certain embodiments, fumaric acid, the fumaric acid ester, dimethyl fumarate, monomethyl fumarate ester or salt thereof is administered in combination with a bronchodilator such as albuterol, formoterol, or levalbuterol or salts thereof.

In certain embodiments, fumaric acid, the fumaric acid ester, dimethyl fumarate, monomethyl fumarate ester or salt thereof is administered in combination with a mucolytic agent such as bromhexine or salts thereof.

In certain embodiments, fumaric acid, the fumaric acid ester, dimethyl fumarate, monomethyl fumarate ester or salt thereof is administered in combination with is administered in combination with an anti-inflammatory agent such as a corticosteroid, fluticasone, or salts thereof.

In certain embodiments, fumaric acid, the fumaric acid ester, dimethyl fumarate, monomethyl fumarate ester or salt thereof is administered in combination with an antibiotic agent such as macrolides, azithromycin, antipseudomonal, fluoroquinolones, ciprofloxacin, levofloxacin, ceftazidime, piperacillin and tazobactam, imipenem, aminoglycosides, aztreonam, tobramycin, colistin, colistimethate sodium, or salt thereof.

In certain embodiments, fumaric acid, the fumaric acid ester, dimethyl fumarate, monomethyl fumarate ester or salt thereof is administered in combination with a cystic fibrosis drug such as lumacaftor, elexacaftor, ivacaftor, tezacaftor, cavosonstat, olacaftor, posenacaftor, galicaftor, navocaftor, deutivacaftor, nesolicaftor, or combinations thereof.

In certain embodiments, fumaric acid, the fumaric acid ester, dimethyl fumarate, monomethyl fumarate ester or salt thereof is administered in combination with other pharmaceutically active agents. These compounds include but are not limited to analgesics, anti-inflammatory drugs, antipyretics, antidepressants, antiepileptics, antihistamines, antimigraine drugs, antimuscarinics, anxiolytics, sedatives, hypnotics, antipsychotics, bronchodilators, anti-asthma drugs, cardiovascular drugs, corticosteroids, dopaminergics, electrolytes, gastro-intestinal drugs, muscle relaxants, nutritional agents, vitamins, parasympathomimetics, stimulants, anorectics, and anti-narcoleptics.

Specific examples of the pharmaceutically active agents that can be adjunctively administered include, but are not limited to, aceclofenac, acetaminophen, atomoxetine, almotriptan, alprazolam, amantadine, amcinonide, aminocyclopropane, amitriptyline, amlodipine, amoxapine, amphetamine, aripiprazole, aspirin, atomoxetine, azasetron, azatadine, beclomethasone, benactyzine, benoxaprofen, bermoprofen, betamethasone, bicifadine, bromocriptine, budesonide, buprenorphine, bupropion, buspirone, butorphanol, butriptyline, caffeine, carbamazepine, carbidopa, carisoprodol, celecoxib, chlordiazepoxide, chlorpromazine, choline salicylate, citalopram, clomipramine, clonazepam, clonidine, clonitazene, clorazepate, clotiazepam, cloxazolam, clozapine, codeine, corticosterone, cortisone, cyclobenzaprine, cyproheptadine, demexiptiline, desipramine, desomorphine, dexamethasone, dexanabinol, dextroamphetamine sulfate, dextromoramide, dextropropoxyphene, dezocine, diazepam, dibenzepin, diclofenac sodium, diflunisal, dihydrocodeine, dihydroergotamine, dihydromorphine, dimetacrine, divalproex, dizatriptan, dolasetron, donepezil, dothiepin, doxepin, duloxetine, ergotamine, escitalopram, estazolam, ethosuximide, etodolac, femoxetine, fenamates, fenoprofen, fentanyl, fludiazepam, fluoxetine, fluphenazine, flurazepam, flurbiprofen, flutazolam, fluvoxamine, frovatriptan, gabapentin, galantamine, gepirone, granisetron, haloperidol, huperzine A, hydrocodone, hydrocortisone, hydromorphone, hydroxyzine, ibuprofen, imipramine, indiplon, indomethacin, indoprofen, iprindole, ipsapirone, ketanserin, ketoprofen, ketorolac, lesopitron, levodopa, lipase, lofepramine, lorazepam, loxapine, maprotiline, mazindol, mefenamic acid, melatonin, melitracen, memantine, meperidine, meprobamate, mesalamine, metapramine, metaxalone, methadone, methadone, methamphetamine, methocarbamol, methyldopa, methylphenidate, methylsalycylate, metoclopramide, mianserin, mifepristone, milnacipran, minaprine, mirtazapine, moclobemide, molindone, morphine, morphine hydrochloride, nabumetone, nadolol, naproxen, naratriptan, nefazodone, neurontin, nomifensine, nortriptyline, olanzapine, olsalazine, ondansetron, opipramol, orphenadrine, oxaflozane, oxaprozin, oxazepam, oxitriptan, oxycodone, oxymorphone, pancrelipase, parecoxib, paroxetine, pemoline, pentazocine, pepsin, perphenazine, phenacetin, phendimetrazine, phenmetrazine, phenylbutazone, phenytoin, phosphatidylserine, pimozide, pirlindole, piroxicam, pizotifen, pizotyline, pramipexole, prednisolone, prednisone, pregabalin, propranolol, propizepine, propoxyphene, protriptyline, quazepam, quinupramine, reboxetine, reserpine, risperidone, ritanserin, rivastigmine, rizatriptan, rofecoxib, ropinirole, rotigotine, salsalate, sertraline, sibutramine, sildenafil, sulfasalazine, sulindac, sumatriptan, tacrine, temazepam, tetrabenazine, thiazides, thioridazine, thiothixene, tiapride, taziprinone, tizanidine, tofenacin, tolmetin, toloxatone, topiramate, tramadol, trazodone, triazolam, trifluoperazine, trimethobenzamide, trimipramine, tropisetron, valdecoxib, valproic acid, venlafaxine, viloxazine, vitamin E, zimeldine, ziprasidone, zolmitriptan, zolpidem, zopiclone, and combinations thereof.

### Pharmaceutical Compositions and Kits

In certain embodiments, this disclosure relates to pharmaceutical composition, containers, and kits comprising fumaric acid, a fumaric acid ester, dimethyl fumarate, monomethyl fumarate ester or salt thereof for use in pulmonary administration and optionally comprising another active agent such as a bronchodilator, corticosteroid, anticholinergic, antimuscarinic, mucolytic agent, anti-inflammatory agent, antibiotic, anti-viral agent, beta-2 agonist, cystic fibrosis drug, or combinations thereof.

In certain embodiments, the pharmaceutical composition is contained in a container comprising an aerosolizing propellant. In certain embodiments, the aerosolizing propellant is compressed air, ethanol, nitrogen, carbon dioxide, nitrous oxide, hydrofluoroalkanes (HFAs), 1,1,1,2,-tetrafluoroethane, 1,1,1,2,3,3,3-heptafluoropropane, chlorofluorocarbon, or combinations thereof.

In certain embodiments, the container is a pressurized or unpressurized container. In certain embodiments, the container is a manual pump spray, inhaler, meter-dosed inhaler, dry powder inhaler, nebulizer, vibrating mesh nebulizer, jet nebulizer, or ultrasonic wave nebulizer.

In certain embodiments, the pharmaceutical composition is an aerosol suspension, a dry powder, or a liquid suspension.

In certain embodiments, the pharmaceutical composition is an inhalation pharmaceutical formulation prepared for delivery as a nasal spray or an inhaler, such as a metered dose inhaler (MDI).

In certain embodiments, the container is a nebulizer comprising fumaric acid, salt, or ester thereof for use in pulmonary administration into a mist, optionally using an aqueous saline solution, inhaled through a mouthpiece or face mask.

In certain embodiments, this disclosure relates to the production of a medicament comprising fumaric acid, a fumaric acid ester, dimethyl fumarate, monomethyl fumarate ester or salt thereof as reported herein for therapeutic uses reported herein.

In certain embodiments, the pharmaceutical composition optionally comprises a pharmaceutical carrier, and that the pharmaceutical composition optionally comprises further therapeutic agents, respiratory agents, anti-inflammatory agents, etc. In certain embodiments, a pharmaceutical composition is in the form of a liquid comprising pH buffering agents and optionally salts and/or saccharide or polysaccharide.

In certain embodiments, this disclosure relates to pharmaceutical compositions comprising fumaric acid, a fumaric acid ester, dimethyl fumarate, monomethyl fumarate ester or salt thereof as reported herein and a pharmaceutically acceptable excipient. In certain embodiments, the pharmaceutically acceptable excipient is selected from lactose, sucrose, mannitol, triethyl citrate, dextrose, cellulose, methyl cellulose, ethyl cellulose, hydroxyl propyl cellulose, hydroxypropyl methylcellulose, carboxymethylcellulose, croscarmellose sodium, polyvinyl N-pyrrolidone, crospovidone, ethyl cellulose, povidone, methyl and ethyl acrylate copolymer, polyethylene glycol, fatty acid esters of sorbitol, lauryl sulfate, gelatin, glycerin, glyceryl monooleate, silicon dioxide, titanium dioxide, talc, corn starch, carnauba wax, stearic acid, sorbic acid, magnesium stearate, calcium stearate, castor oil, mineral oil, calcium phosphate, starch, carboxymethyl ether of starch, iron oxide, triacetin, acacia gum, esters, or salts thereof.

Compositions may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents solvents or vehicles include water, ethanol, polyols (propylene glycol, polyethylene glycol, glycerol, and the like), suitable mixtures thereof, vegetable (such as olive oil, sesame oil) and injectable organic esters such as ethyl oleate.

These compositions may also contain preserving, emulsifying, and dispensing agents. Prevention of the action of microorganisms may be controlled by addition of any of various antibacterial, antiviral, and antifungal agents, example, parabens, chlorobutanol, phenol, sorbic acid, and the like.

Liquid dosage forms may contain inert diluents commonly used in the art, such as water or other solvents, solubilizing agents and emulsifiers, for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil and sesame oil, glycerol, tetrahydrofurfuryl alcohol, polyethylene glycols and fatty acid esters of sorbitan or mixtures of these substances, and the like.

In certain embodiments, the pharmaceutical composition is used as a buccal or nasal spray. In certain embodiments, the pharmaceutical compositions are in a form for inhalation. In certain embodiments, the pharmaceutical composition comprises fumaric acid, a fumaric acid ester, dimethyl fumarate, monomethyl fumarate ester or salt thereof as reported herein and a propellant. In certain embodiments, an aerosolizing propellant is compressed air, ethanol, nitrogen, carbon dioxide, nitrous oxide, hydrofluoroalkanes (HFAs), or combinations thereof.

In certain embodiments, the disclosure contemplates a pressurized or unpressurized container comprising fumaric acid, a fumaric acid ester, dimethyl fumarate, monomethyl fumarate ester or salt thereof as reported herein. In certain embodiments, the container is a manual pump spray, inhaler, meter-dosed inhaler, dry powder inhaler, nebulizer, vibrating mesh nebulizer, jet nebulizer, or ultrasonic wave nebulizer.

In certain embodiments, this disclosure contemplates kits comprising pharmaceutical compositions comprising fumaric acid, a fumaric acid ester, dimethyl fumarate, monomethyl fumarate ester or salt thereof as reported herein and optionally another therapeutic agent in same or separate pharmaceutical composition or container. The kits may contain a transfer device such a needle, syringe, cannula, capillary tube, pipette, or pipette tip.

In certain embodiments, the fumaric acid, a fumaric acid ester, dimethyl fumarate, monomethyl fumarate ester or salt thereof as reported herein may be contained in a storage container, dispensing container, sealed, or unsealed, such a vial, bottle, ampule, blister pack, or box. In certain embodiments, other agents may be contained in a storage container, sealed, or unsealed, such a vial, bottle, ampule, blister pack, or box.

In certain embodiments, the kit further comprises written instructions for using fumaric acid, a fumaric acid ester, dimethyl fumarate, monomethyl fumarate ester or salt thereof as reported herein and optionally other agents for treating and/or preventing a condition in a subject as reported herein.

In certain embodiments, this disclosure relates to uses of fumaric acid, a fumaric acid ester, dimethyl fumarate, monomethyl fumarate ester or salt thereof as reported herein in the production of a medicament for treating conditions disclosed herein.

Dosing is dependent on severity and responsiveness of the disease state to be treated, and the course of treatment lasting from several days to several months, or until a cure is effected or a diminution of the disease state is achieved. Optimal dosing schedules can be calculated from measurements of drug accumulation in the body of the patient. Optimum dosages may vary depending on the relative potency of individual agents. Generally, it can be estimated based on amounts found to be effective in in vitro and in vivo animal models. In general, dosage is from 0.01 μg to 1 g per kg of body weight, and may be given once or more daily, weekly, monthly, or yearly, or even once every 2 to 10 years. Following successful treatment, it may be desirable to have the patient undergo maintenance therapy to prevent the recurrence of the disease state.

### Lung-Targeted Delivery of Dimethyl Fumarate Promotes the Reversal of Age-Dependent Established Lung Fibrosis

Idiopathic pulmonary fibrosis (IPF), a severe and deadly form of lung fibrosis, is widely regarded as a disease of aging. Aged mice with persistent lung fibrosis and IPF lung myofibroblasts exhibit deficient Nrf2-mediated antioxidant responses. Tecfidera™ (dimethyl fumarate) is an orally administered FDA-approved drug for the treatment of multiple sclerosis. The active pharmaceutical ingredient is dimethyl fumarate (DMF), an active Nrf2 activator. Experiments reported herein indicate that in IPF lung fibroblasts, DMF treatment inhibited both TGF-mediated pro-fibrotic phenotypes and led to a reversal of established pro-fibrotic phenotypes.

Experiments were also performed to determine the clinical efficacy of lung-targeted (inhaled) vs. systemic (oral) delivery of DMF in an aging murine model of bleomycin-induced persistent lung fibrosis. DMF or vehicle was administered daily to aged mice by oral gavage or intranasal delivery from 3-6 weeks post-injury when mice exhibited non-resolving lung fibrosis. In contrast to systemic (oral) delivery, only lung-targeted (inhaled) delivery of DMF restored lung Nrf2 expression levels, reduced lung oxidative stress, and promoted the resolution of age-dependent established fibrosis indicating the efficacy of lung-targeted DMF delivery to promote the resolution of age-dependent established lung fibrosis.

Idiopathic pulmonary fibrosis (IPF) is a sometimes fatal and relentlessly progressive disorder characterized by excessive scar tissue formation and irreversible destruction of the lung parenchyma, resulting in gas-exchange abnormalities and ultimately respiratory failure. As the average life expectancy continues to increase, the elderly population is growing at a rapid pace.

The incidence and prevalence of IPF increase with age; two-thirds of IPF patients are older than 60 years at the time of presentation, with a mean age of 66 years at the time of diagnosis. Further, the survival rate for IPF patients markedly decreases with age. Despite this strong association, cellular/molecular mechanisms that account for the aging predilection to fibrotic disease are not well understood. Mechanisms of pathological fibrosis appear to be common among all tissues/organs, in particular oxidative stress and myofibroblast activation. Oxidative stress typically includes an imbalance between reactive oxygen species (ROS) production and antioxidant capacity. Reports indicate defective antioxidant responses in the pathogenesis of IPF. The induction of the nuclear factor erythroid 2-related factor 2 (Nrf2) serves as a master regulator of the cellular antioxidant responses by inducing the transcription of a wide array of genes that can mitigate oxidative stress. However, patients with IPF exhibit decreased Nrf2 expression and redox imbalance that promotes pro-fibrogenic responses in myofibroblasts. In animal models of pulmonary fibrosis, Nrf2 deficiency results in heightened oxidative stress and more severe pulmonary fibrosis.

Experiments indicate that aged mice exhibited an impaired capacity for fibrosis resolution; this is in part regulated by alterations in cellular redox homeostasis resulting from deficient induction of Nrf2 in lung fibroblasts. The efficacy of antioxidant-targeted strategies via Nrf2 activation is shown to suppress the development of bleomycin-induced lung fibrosis in young animals. The efficacy of Nrf2 activation for reversing age-dependent established lung fibrosis has never been evaluated. There is a critical need for antioxidant-based therapies that can promote the resolution of established lung fibrosis without deleterious adverse effects. The goal of certain experiments reported herein was to determine the pre-clinical efficacy of lung-targeted delivery vs. systemic delivery of DMF in an aging murine model of persistent lung fibrosis.

Oxidative stress has long been associated with fibrotic disorders, including IPF. Despite the link between aging and oxidative stress, therapeutically tenable agents that target age-associated oxidative stress to treat fibrosis have yet to be translated into a viable treatment for patients with IPF. Pre-clinical studies demonstrated a critical role for Nrf2 in mediating antioxidant responses during injury-repair. In aged mice, lung injury results in persistent fibrosis, which is associated with defective Nrf2-mediated antioxidant responses. Further, Nrf2 expression was reportedly decreased in the lungs of patients diagnosed with IPF. Nrf2-targeted strategies are yet to be evaluated in clinical trials for IPF. Strategies that more directly target the source(s) of redox imbalance via localized tissue-specific delivery would offer greater potential to reduce unintended side-effects and would be more effective as compared to systemic delivery for reversing age-associated established fibrosis. While systemic (oral) DMF delivery attenuated blood ROS levels, it failed to rescue lung Nrf2 levels and did not demonstrate efficacy for resolving age-dependent established lung fibrosis. Importantly, lung-targeted DMF delivery rescued deficient Nrf2-mediated antioxidant responses within the lung and promoted the resolution of age-dependent established lung fibrosis. This disclosure provides a side-by-side comparison of systemic vs. local delivery of DMF and demonstrates utility for a lung-targeted antioxidant approach as a therapeutic strategy for reversing age-associated established fibrosis.

A limitation of the traditional bleomycin-induced lung fibrosis model in young mice is the resolving nature of fibrosis, as lung injury results in a limited fibrotic response which resolves beyond 3 weeks post-injury. One potential explanation for the limited clinical translation of therapeutics for IPF is that age-dependent pathologic mechanisms remain largely unexploited in the drug development process, despite the fact that aging is strongly implicated in the pathogenesis of IPF. Pre-clinical animal models of lung fibrosis are largely performed in young mice (8-10 weeks), which predominantly results in a self-limited fibrotic response. Treatment interventions are largely preventative (dosing before or at the time of injury) rather than curative. It has become increasingly important to re-evaluate the use of this model for pre-clinical studies to predict therapeutic benefit in subsequent clinical trials, particularly since numerous drug candidates demonstrating efficacy in young mice have failed in clinical trials with elderly patients. This aging murine model of non-resolving lung fibrosis better recapitulates pathological signaling observed in human IPF patients (e.g., defective Nrf2 responsiveness), as limited knowledge of age-dependent pathological mechanisms/targets remain key challenges and may in part explain the limited therapies available. Experiments were performed in an animal model of persistent lung fibrosis to evaluate the pre-clinical efficacy of an Nrf2 activator DMF for reversing established fibrosis (versus evaluating the pre-clinical efficacy of inhibiting de novo synthesis of fibrosis); this is a more clinically relevant efficacy testing protocol since patients with IPF typically present with well-established fibrosis. With the emergence of senolytics as a potential strategy for addressing IPF, age-relevant pre-clinical efficacy models have become increasingly important to enhance the potential for clinical translation.

Gender differences exist in response to bleomycin-induced lung injury; therefore, only female mice were used to avoid sex-related variability of responses to injury. Future studies could further validate these findings in male mice. Leveraging aging models at the appropriate stages of therapeutic development is likely to provide important insights that would accelerate the successful translation of improved therapies for IPF. From the clinical standpoint, lung targeted drug delivery offers several major advantages over systemic delivery routes, including the ability to deliver higher local drug concentration and the potential for reduced side effects. Experiments indicate that lung-targeted delivery of DMF, but not systemic delivery, mediates upregulation of Nrf2 expression, antioxidant activity, and promotes partial resolution of age-dependent established lung fibrosis. Of note, although the oral dose was three times higher (12 mg/kg) than the inhaled dose (4 mg/kg), only inhaled delivery demonstrated efficacy for promoting reversal of established fibrosis, despite the lower dose. Thus, although current IPF therapies are administered orally, lung-targeted delivery of novel therapeutics may provide greater efficacy (even at lower doses with reduced side effects). No therapeutics have been shown to reverse age-associated established fibrosis, which may represent the holy grail for therapeutic strategies to more effectively treat IPF.

### Experiments on Therapeutic Treatments

At 3 weeks post-bleomycin administration, mice were randomly assigned to one of four treatment groups. Between 3-6 weeks post-injury, DMF or vehicle (sterile PBS) was administered daily by oral gavage (240 μg/dose in 150 μL total volume) (Table 1) using a gavage tube. In parallel, mice were anesthetized using isoflurane and administered DMF or vehicle (sterile PBS) daily by intranasal instillation (80 μg/dose in 50 μL total volume) (Table 1). The control animals received vehicle only. Dosing was based on the solubility of this compound in the vehicle in the total volume used for administration (DMF was dissolved in sterile PBS at 42° C. and sonicated for 50 min). Mice were monitored for body-weight changes and survival. Mice were removed from the study when greater than 20% weight loss was observed compared to pre-surgery (day 0) or lack of responsiveness to touch. At 6 weeks post-injury, mice were sacrificed by CO₂ inhalation.

TABLE 1
DMF Formulation Protocol

| Delivery Route | Vehicle | Volume | | Dose | |
|---|---|---|---|---|---|
| Intranasal | PBS | 50 | μL | 80 | μg |
| Oral gavage | PBS | 150 | μL | 240 | μg |

### DMF Treatment Reduces ROS Levels in IPF Lung Fibroblasts

In IPF lung fibroblasts, decreased Nrf2 expression was associated with profibrogenic phenotypes. Further, Nrf2 activation (via sulforaphane treatment) restored antioxidant responses and promoted dedifferentiation of IPF myofibroblast. IPF lung myofibroblasts exhibit defective Nrf2 responses associated with oxidative stress. Since primary mesenchymal cells isolated from IPF lungs represent a heterogeneous population of both fibroblasts and myofibroblasts “(myo)fibroblasts”, the efficacy of DMF was evaluate on both TGF-beta-induced and established profibrogenic phenotypes of these cells. To determine if DMF treatment can inhibit fibrogenic responses induced by TGF-beta, cells were pre-treated with DMF or vehicle followed by treatment with TGF-beta for 24 h. DMF treatment significantly reduced TGF-beta-induced production of ROS, specifically H₂O₂, as compared to vehicle treatment. DMF treatment also led to the inhibition of TGF-beta-induced expression of collagen-1, a major ECM component in IPF fibroblasts. Since primary IPF lung (myo) fibroblasts exhibit enhanced production of ROS and ECM, experiments were performed to determine if DMF can reverse these established pro-fibrotic phenotypes. DMF treatment significantly inhibited the production of H₂O₂ and collagen-1, as compared to vehicle treatment. These results support the concept that activation of Nfr2 by DMF could be a therapeutic approach to target redox imbalance and reduce fibrogenic responses in IPF.

### Oral DMF Delivery Fails to Reverse Age-Dependent Persistent Lung Fibrosis

In young mice, bleomycin-induced injury results in a self-limited fibrotic response, where fibrosis spontaneously resolves following peak injury. Conversely, aged mice exhibit a persistent fibrotic response, with little to no resolution of fibrosis from 3 weeks to 4 months post-injury. Nrf2 levels are highly upregulated in young mice following bleomycin-induced injury, which promotes fibrosis resolution. In contrast, the lack of fibrosis resolution in aged mice is associated with a deficient Nrf2 response, where Nrf2 levels are significantly downregulated. Experiments were performed to determine whether aged mice exhibit a deficient Nrf2 response following bleomycin-induced lung injury. Aged mice demonstrated a significant decrease in lung Nrf2 expression levels at 3 weeks post-injury, which was accompanied by increased levels of oxidized glutathione, and increased expression of ECM markers, including fibronectin and collagen-1.

This aging model of bleomycin-induced persistent lung fibrosis enables the evaluation of therapeutic agents for their efficacy in reversing age-dependent established fibrosis. Therefore, experiments were performed to determine whether systemic delivery of DMF (via oral gavage) demonstrates efficacy for reversing age-dependent established fibrosis. Starting at 3 weeks post-injury (when aged mice exhibit established/persistent fibrosis), DMF or vehicle was administered by oral gavage daily through 6 weeks. Aged vehicle-treated mice demonstrated persistent lung fibrosis at 6 weeks post-injury, as demonstrated by histopathology, lung collagen deposition, elevated fibronectin, and collagen-1 expression levels, and elevated lung hydroxyproline levels. However, oral DMF treatment failed to promote the resolution of age-dependent established lung fibrosis during this 3-week treatment period, as lung fibrosis in mice treated with oral DMF was indistinguishable from vehicle-treated mice. Overall, systemic (oral) administration of DMF failed to reverse age-dependent established lung fibrosis.

### Intranasal DMF Delivery Promotes Resolution of Age-Dependent Established Fibrosis

Inhaled drug delivery offers advantages over systemic (oral) drug administration for lung-targeted indications, as higher drug concentrations can be delivered directly to the lungs while potentially reducing the concentration of systemic exposure among other organs. Therefore, experiments were performed to evaluate the efficacy of lung-targeted DMF treatment (via inhaled delivery) for reversing age-dependent established fibrosis (as opposed to systemic delivery, which failed to demonstrate efficacy). Using the same injury model and treatment protocol, aged mice were administered DMF or vehicle daily via intranasal delivery from 3-6 weeks post-injury. Inhaled DMF treatment resulted in resolution of age-dependent established lung fibrosis, as demonstrated by histopathology, reduced collagen deposition, significantly decreased expression levels of fibronectin and collagen-1, and decreased lung hydroxyproline levels as compared to vehicle-treated and oral DMF treated mice. These data suggest that, although systemic DMF delivery failed to reverse established fibrosis, lung-targeted DMF treatment via inhaled delivery demonstrated efficacy for promoting the resolution of age-dependent established lung fibrosis.

### Intranasal DMF Delivery Rescues Deficient Nrf2-Mediated Antioxidant Responses and Promotes Lung Redox Homeostasis

Given that lung-targeted inhaled delivery of DMF demonstrated efficacy for promoting the resolution of age-dependent established lung fibrosis, whereas systemic oral delivery failed to demonstrate efficacy, the pharmacodynamics of oral vs. inhaled DMF delivery was evaluated. Although oral administration of DMF led to reduced blood ROS levels, it failed to rescue deficient Nrf2 expression levels in the lungs of aged mice with established fibrosis. In contrast, although intranasal DMF delivery did not impact blood ROS levels, it resulted in significantly increased lung Nrf2 expression levels. Further, intranasal DMF delivery also led to significantly reduced oxidized glutathione levels and lipid peroxidation in the lungs, as compared to vehicle or oral DMF treatment. These data suggest that inhaled DMF delivery promotes Nrf2-mediated lung antioxidant responses to rescue lung redox imbalance without affecting systemic redox levels. In summary, lung-targeted treatment with DMF via inhaled delivery (as compared to oral delivery) can more effectively rescue local Nrf2 expression levels and promote redox homeostasis in the lungs.

What is claimed is:

1. A method of treating or preventing lung fibrosis comprising administering an effective amount of dimethyl fumarate by inhalation through a nostril or mouth to the lungs to a human subject in need thereof.

2. The method of claim 1, wherein the subject is older than 55 years old.

3. The method of claim 1, wherein the subject is diagnosed with pulmonary fibrosis.

4. The method of claim 1, wherein the subject is diagnosed with bronchiectasis.

5. The method of claim 1, wherein administration is by a nebulizer.

6. The method of claim 5, wherein nebulizer is a jet nebulizer driven by compressed air.

7. The method of claim 5, wherein the nebulizer is an ultrasonic nebulizer having a piezoelectric transducer for creating droplets from a liquid reservoir.

8. The method of claim 5, wherein the nebulizer is vibrating mesh nebulizer having perforated membranes actuated by an annular piezo element to vibrate.

9. The method of claim 1, wherein administration is by a pressurized or unpressurized inhaler.

10. The method of claim 1, wherein administration is by a metered-dose inhaler.

11. The method of claim 1, wherein the subject is diagnosed with bacterial infection.

12. The method of claim 1, wherein dimethyl fumarate is administered in combination with a bronchodilator.

13. The method of claim 1, wherein dimethyl fumarate is administered in combination with nintedanib.

14. The method of claim 1, wherein dimethyl fumarate is administered in combination with pirfenidone.

15. The method of claim 1, wherein dimethyl fumarate is administered in combination with anti-inflammatory agent.

16. The method of claim 1, wherein dimethyl fumarate is administered in combination with an antibiotic agent.

17. A pharmaceutical container comprising dimethyl fumarate wherein the container is an inhaler, metered-dose inhaler, manual pump spray, nebulizer, vibrating mesh nebulizer, jet nebulizer, or ultrasonic wave nebulizer.

18. The pharmaceutical container of claim 17 comprising an aerosolizing propellant.

19. A pharmaceutical composition comprising dimethyl fumarate and another active agent.

20. The pharmaceutical composition of claim 19, wherein the active agent is a bronchodilator, corticosteroid, anticholinergic, antimuscarinic, mucolytic agent, anti-inflammatory agent, antibiotic, beta-2 agonist, cystic fibrosis drug, or combinations thereof.
